(19) United States
(12) Patent Application Publication
(10) Pub. No.: US 2011/0220121 A1
(43) Pub. Date: Sep. 15, 2011

(54) Full male genital coverage STD protection and contraception condom

(76) Inventor: Jennifer Anne Holley, Taylor, MI (US)

(21) Appl. No.: 12/925,097

(22) Filed: Mar. 11, 2010

(51) Int. Cl. A61F 6/04

(52) U.S. Cl. 128/844

(57) ABSTRACT

The male condom is designed to provide significantly improved STD protection for both male and female partners. In addition, the condom provides barrier contraceptive birth control for the female (FIG. 1). The scrotal pouch (A) is molded in one piece together with the rest of the condom for coverage of the entire male genitalia. The teat end of the condom (B) provides a semen reservoir. The condom will be manufactured in two sizes. Normal size: (D)=52 mm (2.0 inches) and (E)=190 mm (7.4 inches). Extra large size: (D)=(56 mm (2.2 inches) and (e)=210 mm (8.2 inches). The opening (b) has a beaded cuff for strength and easy application measuring 25 mm (1.0 inches) non-stretched. The condom will be manufactured in biodegradable natural latex. The condom would be manufactured in normal strength and extra strength as well as lubricated and non-lubricated versions.

### CROSS-REFERENCE TO RELATED APPLICATIONS

N/A

### STATEMENT REGARDING FEDERALLY SPONSORED RESEARCH

N/A

### BACKGROUND OF THE INVENTION

Male condoms are currently available on the market in many different varieties. There are latex versions, lamb skin versions, lubricated, non-lubricated, extra large and normal size. Currently there are no versions available that offer protection to the male scrotal sac. It is my intention to provide a condom that will increase STD protection for both male and female partners while at the same time continues to offer contraception protection.

### SUMMARY OF THE INVENTION

The full male genital coverage STD protection and contraception condom is claimed to provide significantly improved STD protection for both male and female partners above that of a normal design male condom. In addition, the condom is clamed to provide barrier contraceptive birth control for the female.

### DESCRIPTION OF THE DRAWINGS

The scrotal pouch (A) is molded in one piece together with the rest of the condom, thereby integrating the scrotal pouch into the penile shaft to ensure seamless, complete and reliable barrier coverage of the entire male genitalia. The teat end feature of the condom (C) provides a reservoir for ejaculated semen adding comfort and safety for both the male and female partners. The condom is expected to be manufactured in two sizes, a normal size for the average size male penis and an extra large size for the larger than average male penis. The normal size condom measurements are: (D)=52 mm (2.0 inches) and (E)=190 mm (7.4 inches). The extra large condom measurements are: (D)=56 mm (2.2 inches) and (E)=210 mm (8.2 inches). The opening (B) has a beaded cuff for strength and easy application measuring 25 mm (1.0 inches) non-stretched allowing for a snug fit round the posterior scrotum and penile shaft base thereby covering the entire male genitalia.

### DETAILED DESCRIPTION OF THE INVENTION

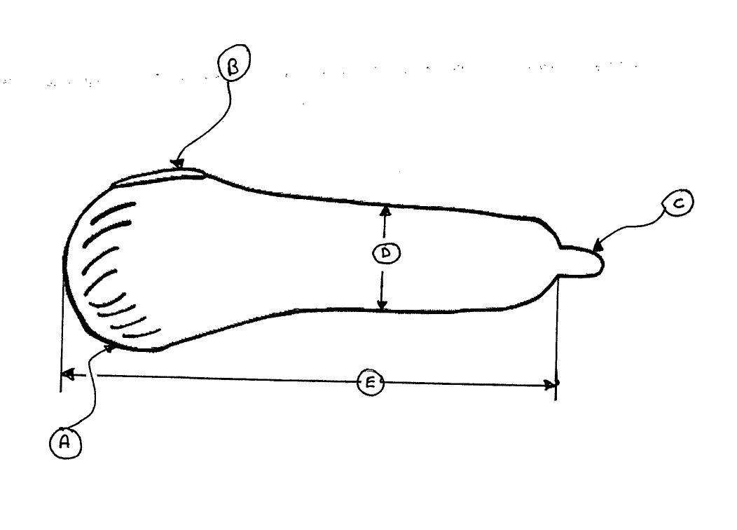

The male condom is designed to provide significantly improved STD protection for both male and female partners above a normally design male condom. In addition, the condom provides barrier contraceptive birth control for the female (FIG. 1). The scrotal pouch (A) is molded in one piece together with the rest of the condom thereby integrating the scrotal pouch into the penile shaft to ensure seamless, complete and reliable barrier coverage of the entire male genitalia. The teat end of the condom (B) provides a reservoir for ejaculated semen adding comfort and safety for both the male and female partners. The condoms is expected to be manufactured in two sizes, a normal size for the average size male penis and an extra large size for the larger than average male penis. The normal size condom measurements are: (D)=52 mm (2.0 inches) and (E)=190 mm (7.4 inches). The extra large condom measurements are: (D)=(56 mm (2.2 inches) and (e)=210 mm (8.2 inches). The opening (b) has a beaded cuff for strength and easy application measuring 25 mm (1.0 inches) non-stretched allowing for a snug fit around the posterior scrotum and penile shaft base thereby covering the entire male genitalia.

It is expected that the condom would be manufactured in a biodegradable natural latex material making the condom environmentally friendly. It is also expected that the condom would be manufactured in normal strength and extra strength as well as lubricated and non-lubricated versions.

1. The above named condom is claimed to to provide significantly improved STD protection for both male and female partners above a normal design male condom. In addition, the condom is claimed to provide barrier contraceptive birth control for the female.
The scrotal pouch (A) is moulded in one piece together with the rest of the condom thereby integrating the scrotal pouch into the penile shaft to ensure seamless, complete and reliable barrier coverage of the entire male genitalia. The teat end feature of the condom (B) provides a reservoir for ejaculated semen adding comfort and safety for both the male and female partners. The condom is expected to be manufactured in two sizes, a normal size for the average size male penis and an extra large size for the larger than average male penis. The normal size condom measurements are: (D)=52 mm (2.0 inches) and (E)=190 mm (7.4 inches). The extra large condom measurements are: (D)=56 mm (2.2 inches) and (E)=210 mm (8.2 inches). The opening (B) has a beaded cuff for strength and easy application measuring 25 mm (1.0 inches) non-stretched allowing for a snug fit round the posterior scrotum and penile shall base thereby covering the entire male genitalia.
